(19) United States
(12) Patent Application Publication
(10) Pub. No.: US 2026/0097100 A1
(43) Pub. Date: Apr. 9, 2026

(54) CANCER TREATMENT BY INDUCTION OF TUMOR AND STEM CELL DIFFERENTIATION

(71) Applicants: Keith Roger Latham, Kingsport, TN (US); Regina L. Garland, Kingsport, TN (US)

(72) Inventors: Keith Roger Latham, Kingsport, TN (US); Regina L. Garland, Kingsport, TN (US)

(21) Appl. No.: 19/340,296

(22) Filed: Sep. 25, 2025

Related U.S. Application Data
(60) Provisional application No. 63/704,616, filed on Oct. 8, 2024.

(51) Int. Cl. A61K 38/22; A61K 9/00; A61K 47/20; A61P 35/00

(52) U.S. Cl. CPC A61K 38/22; A61K 9/0014; A61K 47/20; A61P 35/00

(57) ABSTRACT

A method and composition for treating cancer through induction of tumor cell differentiation is disclosed. Cancer cells are converted into terminally differentiated, non-proliferating cell types using specific differentiation-inducing agents, including thyroid hormone analogs such as T2 and RT3. The approach avoids the cytotoxic effects and side effects associated with conventional chemotherapy while potentially providing curative treatment. Pharmaceutical compositions include topical formulations for skin cancer treatment and various delivery systems for systemic administration. In vitro screening methods for identifying differentiation-inducing agents are also provided.

### CROSS-REFERENCE TO RELATED APPLICATION

This application claims the benefit of U.S. Provisional Patent Application No. 63/704,616, filed Oct. 8, 2024, titled “Cancer Treatment by Induction of Tumor Cell Differentiation,” which is incorporated herein by reference in its entirety.

### FIELD OF THE INVENTION

The present invention relates to methods and compositions for treating cancer through the induction of tumor cell differentiation, wherein cancer cells are converted into terminally differentiated, non-proliferating cell types, thereby providing therapeutic benefit without the adverse side effects associated with conventional cytotoxic chemotherapy.

### BACKGROUND OF THE INVENTION

Cancer remains one of the leading causes of death globally, with conventional treatments presenting significant challenges. While the immune system can naturally identify and eliminate aberrant cells, cancer cells possess sophisticated mechanisms to evade immune surveillance. Current treatments, particularly chemotherapy, utilize cytotoxic agents to target and destroy cancer cells but often result in collateral damage to healthy cells, causing severe side effects that can limit both quality of life and treatment success.

The present invention addresses these limitations by providing a novel therapeutic approach that harnesses natural cellular differentiation mechanisms to redirect cancer cells away from their proliferative state and into terminally differentiated, non-cancerous cell types.

### Age-Related Cancer Incidence

Cancer incidence demonstrates a striking age-related pattern, as shown in Table 1 below:

TABLE 1
Death Rate from Cancer by Age

| Age (years) | Death Rate/100,000 | Multiple |
|---|---|---|
| <5 | 5.2 | 1 |
| 5-14 | 3.6 | 1 |
| 15-49 | 25 | 5 |
| 50-69 | 270.8 | 62 |
| 70+ | 974.5 | 221 |

Data Source: IHME, Global Burden of Disease (2024)

This age-related pattern suggests that differentiation signals present during embryonic development and early life may be diminished with aging, contributing to increased cancer susceptibility.

### Cellular Differentiation in Development

During normal human development, a fertilized oocyte undergoes a series of divisions, forming specialized cells and tissues essential for proper bodily function. This process is mediated by complex signaling cascades that direct cellular differentiation. In some cases, certain cells may retain a proliferative, stem-cell-like state, which can potentially lead to malignant transformation.

The cellular differentiation signals active during development and early life represent a therapeutic opportunity. Evidence from populations with lower cancer rates, such as “Blue Zones,” suggests that certain natural compounds consumed in the diet may provide differentiation signals that help maintain cellular homeostasis.

### SUMMARY OF THE INVENTION

The present invention provides methods and compositions for treating cancer through the induction of tumor cell differentiation. The invention encompasses:
Methods for inducing cancer cell differentiation using specific signaling compounds, including but not limited to thyroid hormones and their analogs, hormones, growth factors, and natural products.
Pharmaceutical compositions containing differentiation-inducing agents formulated for various delivery routes including oral, sublingual, topical, intravenous, and intraperitoneal administration.
Treatment protocols for various cancer types based on tumor-specific differentiation requirements.
In vitro screening methods for identifying and characterizing differentiation-inducing agents.

The therapeutic approach of the present invention offers significant advantages over conventional cancer treatments by:
Converting cancer cells into harmless, terminally differentiated cells
Avoiding the cytotoxic effects of chemotherapy
Reducing treatment-related side effects
Potentially providing curative treatment for various cancer types

These and other features, aspects and advantages of the present invention will become better understood with reference to the following drawings, description and claims.

### BRIEF DESCRIPTION OF THE DRAWINGS

FIG. 1 shows microscopic images of cultured HeLa cells treated with thyroid hormone analogs T2 and T3, demonstrating morphological changes consistent with cellular differentiation;

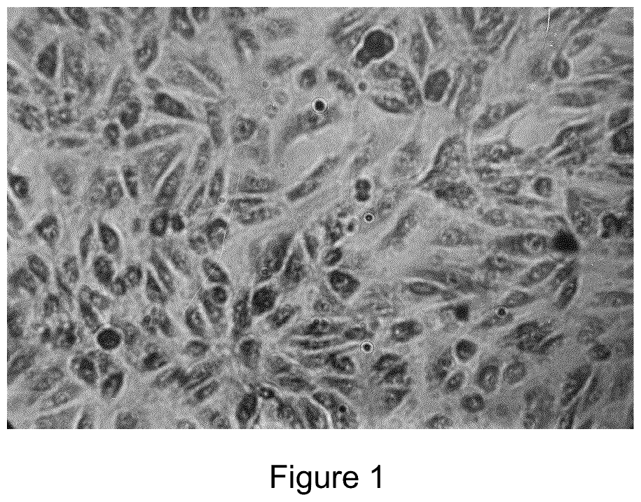

FIG. 2 illustrates clinical results of skin cancer treatment using Carcinin® cream containing T2 (3,5-Diiodo-L-Thyronine), showing therapeutic response in treated lesions; and

FIG. 3 illustrates clinical results of skin cancer treatment using Carcinin® cream containing T2 (3,5-Diiodo-L-Thyronine), showing therapeutic response in treated lesions.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

### I. Theoretical Framework

The present invention is based on the principle that cancer cells can be redirected from their proliferative state into terminally differentiated, non-dividing cell types through appropriate signaling. This approach leverages the natural cellular differentiation mechanisms that operate during embryonic development and tissue homeostasis.

Cancer cells often retain characteristics of undifferentiated or stem-like cells, including:
Unlimited proliferative potential
Resistance to apoptosis
Metabolic reprogramming
Evasion of immune surveillance

By inducing differentiation, these malignant characteristics can be reversed, converting cancer cells into:
Terminally differentiated, post-mitotic cells
Cells responsive to normal growth controls
Cells susceptible to normal apoptotic pathways
Cells recognized by the immune system

### II. Differentiation-Inducing Agents

The present invention encompasses various classes of compounds capable of inducing cancer cell differentiation:

### A. Thyroid Hormones and Analogs

T2 (3,5-Diiodo-L-Thyronine): Demonstrated efficacy in HeLa cell differentiation and topical skin cancer treatment
T3 (Triiodothyronine): Effective in inducing cellular differentiation in vitro
RT3 (Reverse T3, 3,3′,5′-Triiodo-L-Thyronine): Anti-inflammatory properties with tumor growth inhibition
T4 (Thyroxine) and related analogs

### B. Hormones and Growth Factors

Insulin
Apo-Transferrin
EGF (Epidermal Growth Factor)
FGF (Fibroblast Growth Factor)
Hydrocortisone
Steroids (Testosterone, Progesterone, Estrogen, Cortisol)
Prolactin
Corticosterone

### C. Natural Products and Vitamins

Ascorbic Acid (Vitamin C)
Resveratrol
Melatonin
Alpha-Lactalbumin
Folate
Nicotinamide compounds (NADH, NAD+, NR)
Lipoic Acid
Hyaluronic Acid

### D. Synthetic and Pharmaceutical Agents

DMSO (Dimethyl sulfoxide)
Fenbendazole
Rapamycin
Amiodarone and Desethylamiodarone
Tirzepatide
Various antibiotics

### III. Pharmaceutical Compositions and Formulations

The differentiation-inducing agents of the present invention can be formulated into various pharmaceutical compositions suitable for different administration routes:

### A. Topical Formulations

### Example Formulation—Carcinin® Skin Cream (Protocol 2087):

Active Ingredient: T2 (3,5-Diiodo-L-Thyronine)—800 mg
DMSO—220 ml
Transdermal Cream Base—580 gm
Total batch size: 800 gm

### Preparation Method:

Prepare DMSO/T2 solution in appropriate vessel
Add transdermal base to DMSO solution
Mix thoroughly using appropriate blending equipment
Heat to approximately 50° C. to ensure homogeneity
Continue mixing until uniform consistency achieved
Dispense into appropriate containers

### B. Alternative Topical Formulation

### RT3 Skin Cream (Protocol 2089):

Active Ingredient: RT3 (3,3′,5′-Triiodo-L-Thyronine)—800 mg
DMSO—220 ml
Transdermal Cream Base—580 gm

### C. Other Dosage Forms

Oral tablets and capsules
Sublingual tablets
Injectable solutions (IV, IP)
Sustained-release formulations
Liposomal preparations
Nanoparticle formulations

### IV. Methods of Treatment

### A. General Treatment Protocol

1. Patient Assessment: Determine cancer type, stage, and differentiation potential
2. Agent Selection: Choose appropriate differentiation-inducing agent(s) based on tumor characteristics
3. Formulation Selection: Select optimal delivery method and formulation
4. Treatment Administration: Apply/administer according to established protocols
5. Monitoring: Assess treatment response through appropriate biomarkers and imaging
6. Adjustment: Modify treatment regimen based on response

### B. Tumor-Specific Approaches

Different cancer types may require specific differentiation protocols:
Melanoma: Differentiation into fibroblasts using thyroid hormone analogs
Skin Cancers: Topical application of T2, T3 or RT3 formulations
Epithelial Cancers: Differentiation into normal epithelial cell types
Hematological Cancers: Induction of terminal differentiation in blood cell lineages

### C. Combination Therapies

The invention encompasses combination treatments using:
1. Multiple differentiation agents
2. Sequential treatment protocols
3. Combination with conventional therapies where appropriate
4. Adjuvant treatments to enhance differentiation

### V. In Vitro Methods and Screening

### A. Cell Culture Systems

The invention includes methods for screening and characterizing differentiation-inducing agents using established cell culture systems:

### HeLa Cell Model (Example 1):

Culture HeLa cells in appropriate serum-free medium
Treat with test compounds at various concentrations
Monitor cellular morphology and proliferation
Assess differentiation markers
Quantify conversion to differentiated cell types

### B. Screening Protocols

1. Primary Screening: Test compounds for growth inhibition
2. Differentiation Assessment: Evaluate morphological changes
3. Molecular Analysis: Measure differentiation-specific markers
4. Functional Studies: Assess loss of malignant characteristics
5. Dose-Response Analysis: Determine optimal treatment concentrations

### VI. Clinical Applications

### A. Patient Selection Criteria

Confirmed cancer diagnosis
Tumors expressing appropriate differentiation targets
Adequate performance status
No contraindications to specific agents

### B. Treatment Monitoring

Regular assessment of tumor response
Monitoring for treatment-related effects
Biomarker analysis for differentiation progress
Imaging studies to assess tumor status

### C. Safety Considerations

Generally improved safety profile compared to cytotoxic chemotherapy
Agent-specific precautions and contraindications
Regular monitoring for any adverse effects
Dose adjustments based on patient response

### EXAMPLES

### Example 1: HeLa Cell Differentiation In Vitro

HeLa cells (human cervical cancer cell line) were cultured in EX-CELL HeLa Serum-Free Medium (Sigma #14591C). Cells were treated with 1 nM concentrations of either T3 (triiodothyronine) or T2 (3,5-diiodo-L-thyronine) added to the culture medium.

Results: Treatment with either T3 or T2 resulted in:
Significant reduction in cell proliferation
Morphological changes consistent with differentiation
Conversion to fibroblast-like cell morphology
Loss of characteristics associated with malignant transformation

These results demonstrate the feasibility of inducing cancer cell differentiation using thyroid hormone analogs.

### Example 2: Topical Treatment of Skin Cancer

Patients with various types of skin cancer were treated with Carcinin® cream containing T2 (3,5-Diiodo-L-Thyronine) as the active ingredient. The cream was formulated as described in Protocol 2087 and applied topically to affected areas.

### Treatment Protocol:

Daily application to affected skin lesions
Treatment duration varied based on lesion response
Regular photographic documentation of treatment progress

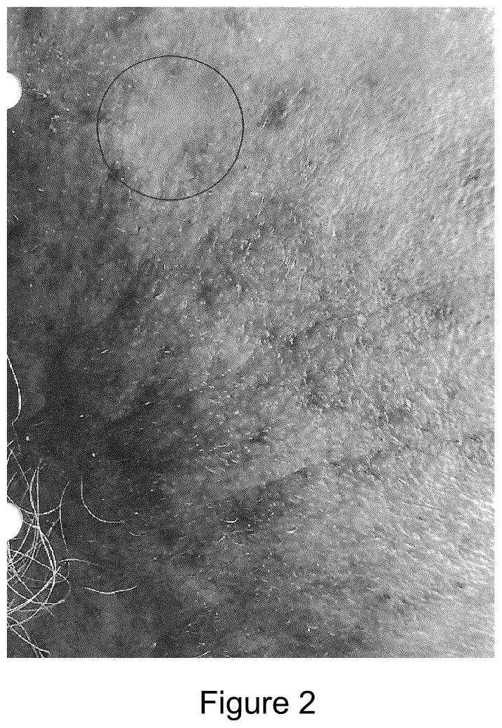

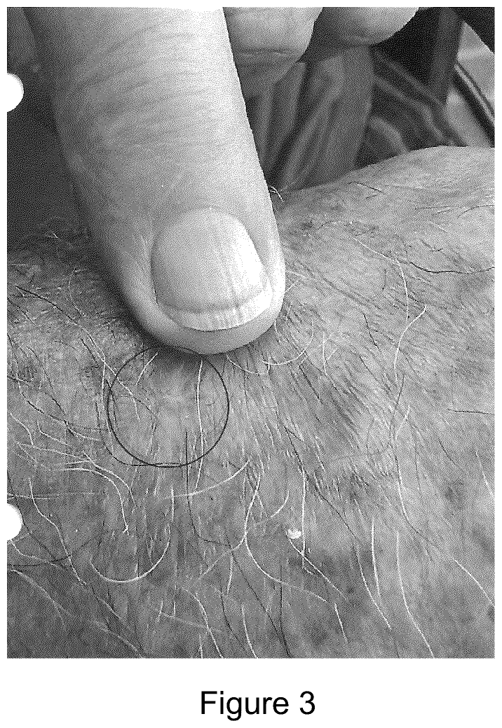

Results (as shown in FIGS. 2 and 3):
Visible reduction in lesion size and appearance
Improved skin texture and appearance
No significant adverse effects reported
Evidence of tissue normalization

### Example 3: RT3 Anti-Inflammatory Treatment

RT3 (Reverse T3) cream was prepared according to Protocol 2089 and evaluated for its anti-inflammatory and anti-proliferative effects on tumor cells.

### Formulation:

RT3 (3,3′,5′-Triiodo-L-Thyronine)—800 mg per 800 gm batch
DMSO carrier system for enhanced penetration
Transdermal cream base for optimal delivery

Application: The RT3 formulation was designed to control local and systemic inflammation that may contribute to tumor cell proliferation and cancer progression.

It should be understood, of course, that the foregoing relates to exemplary embodiments of the invention and that modifications may be made without departing from the spirit and scope of the invention as set forth in the following claims.

What is claimed is:

1. A method for treating cancer comprising administering to a patient in need thereof a therapeutically effective amount of a differentiation-inducing agent capable of converting cancer cells into terminally differentiated, non-proliferating cells.

2. The method of claim 1, wherein the differentiation-inducing agent is selected from the group consisting of thyroid hormones, thyroid hormone analogs, growth factors, hormones, natural products, and synthetic compounds.

3. The method of claim 2, wherein the thyroid hormone analog is selected from the group consisting of T2 (3,5-Diiodo-L-Thyronine), T3 (Triiodothyronine), T4 (Thyroxine), and RT3 (3,3′,5′-Triiodo-L-Thyronine).

4. The method of claim 1, wherein the cancer cells are converted into fibroblasts, epithelial cells, or other terminally differentiated cell types.

5. The method of claim 1, wherein the administration route is selected from the group consisting of topical, oral, sublingual, intravenous, and intraperitoneal.

6. The method of claim 1, wherein the treatment results in cancer cell conversion without the cytotoxic side effects associated with conventional chemotherapy.

7. The method of claim 1, wherein the differentiation-inducing agent is selected from the group consisting of: Insulin, Apo-Transferrin, EGF, FGF, Hydrocortisone, steroids, Ascorbic Acid, Resveratrol, DMSO, Fenbendazole, Nicotine, Folate, Corticosterone, Melatonin, NADH, NAD+, Nicotinamide Riboside, Alpha-Lactalbumin, Prolactin, Amiodarone, Rapamycin, Tirzepatide, Hyaluronic Acid, and Lipoic Acid.

8. A pharmaceutical composition comprising a differentiation-inducing agent and a pharmaceutically acceptable carrier, wherein the differentiation-inducing agent is capable of inducing cancer cell differentiation.

9. The pharmaceutical composition of claim 8, formulated for topical administration and comprising T2 (3,5-Diiodo-L-Thyronine) in a transdermal cream base with DMSO.

10. The pharmaceutical composition of claim 8, formulated for topical administration and comprising RT3 (3,3′,5′-Triiodo-L-Thyronine) in a transdermal cream base with DMSO.

11. A method for screening differentiation-inducing agents comprising:
a) culturing cancer cells in vitro;
b) treating the cancer cells with test compounds;
c) monitoring cellular proliferation and morphological changes;
d) identifying compounds that induce conversion to terminally differentiated cell types.

12. The method of claim 11, wherein the cancer cells are HeLa cells and the test compounds include thyroid hormones and analogs thereof.

13. The method of claim 1, wherein therapeutic agents like thyroid hormones are used, directly or indirectly, modifying the pattern of gene expression in target cells, including cancer and stem cells.
